(19) United States
(12) Patent Application Publication
(10) Pub. No.: US 2007/0004993 A1
(43) Pub. Date: Jan. 4, 2007

(54) Reinforced low temperature thermoplastic material

(75) Inventors: Daniel D. Coppens, Avondale, PA (US); John Damon Kirk, Ramsey, NJ (US); Christopher Wonderly, Exton, PA (US)

Correspondence Address: GOMEZ INTERNATIONAL PATENT OFFICE, LLC, 1501 N. RODNEY STREET, SUITE 101, WILMINGTON, DE, 19806, US

(73) Assignee: QFIX Systems, LLC, Avondale, PA (US)

(21) Appl. No.: 11/368,991

(22) Filed: Mar. 6, 2006

Related U.S. Application Data
(60) Provisional application No. 60/658,618, filed on Mar. 4, 2005.

(51) Int. Cl. A61F 5/00; A61F 5/37

(52) U.S. Cl. 602/7; 128/846

(57) ABSTRACT

A low temperature thermoplastic material for use in medical procedures including radiotherapy patient immobilization, orthopedic splinting or casting, plastic and reconstructive surgery splinting, and orthotic or prosthetic socket cone production or reproduction. The material is made from a thermoplastic that softens when heated to approximately 140 F, after which it can then be formed directly on the patient. The material will then retain this new shape as it cools. The material is composed of Polycaprolactone reinforced with a discontinuous short length fiber and/or fines. The device can also be cross-linked to improve its handling properties.

### PRIORITY CLAIM

This application claims the benefit of U.S. Provisional Application 60/658,618, filed 4 Mar. 2005 and U.S. Provisional application 60/719,143 filed 21 Sep. 2005.

### BACKGROUND OF THE INVENTION

Low temperature thermoplastics have long been used to immobilize or position patients or patient body parts during or after various medical procedures. These medical procedures include, but are not limited to, radiotherapy patient immobilization, orthopedic casting or splinting, plastic and reconstructive surgery splinting, and orthotic or prosthetic socket cone production or reproduction. Aquaplast, a low temperature thermoplastic material invented by WFR/Aquaplast Corp and covered in U.S. Pat. No. 4,240,415, is a similar material to the current invention that has long been used for these types of applications. However, there is a need for low temperature materials that have increased stiffness yet are comfortable against the patient's skin and are easily moldable.

### SUMMARY OF THE INVENTION

The current invention solves the above described need and has many of the same molding and handling characteristics as Aquaplast, but when reinforced with short length fibers, it can be up to 30 times stiffer in the hardened state. This increased stiffness is a very desirable trait as it will either immobilize the patient body part in a more reproducible manner (or in a more protected position depending on the application) or it will allow a thinner piece of thermoplastic to be used with the same level of reproducibility or protection. The fiber reinforcement will also increase the durability of the invention when compared to the prior art.

Specifically, the present invention teaches a cross-linked thermoplastic polyester having a melting point between 50 degrees Celsius and 85 degrees Celsius which is reinforced with a discontinuous short length fiber and or fines.

In a preferred embodiment an aramid fiber can be used to reinforce electron beam cross-linked polycaprolactone having a weight average molecular weight of greater than 5,000. In our tests short length aramid fiber and/or fines worked particularly well for this application. Short length aramid fiber is sold by Dupont under the brand name Kevlar®. It should be noted that fibers of shorter length than 0.25 mm are not readily commercially available, however if they were available they would most likely also work well for this application. Aramid fines, which are shorter than 0.25 mm, are commercially available and have been found to add stiffness to the material.

A variety of other fibers can also be used such as Vectran®, Spectra®, Dyneema ®, fiberglass, carbon fiber and several natural fibers. For medical applications, it is often desirable for the fiber to be stiff, radiolucent and soft against the skin. For this reason, fibers such as aramid fibers and ultra high molecular weight (UHMW) Polyethylene are particularly well suited to this application.

One desirable trait of a material used in these applications is the ability to stretch to at least 150% of its original length when softened. This requirement reduces the marketability of more obvious combinations of reinforcing fibers with a splinting material as the fiber generally prevents stretching of the material when softened. We have produced aramid reinforced polycaprolactone sheets with an 800% maximum elongation. This maximum elongation is comparable with certain grades of Aquaplast.

One problem we discovered when adding fiber to polycaprolactone is that many fibers that could be used to stiffen the material also cause the material to have a rough surface. The rough surface texture is magnified as the material is stretched. Aramid fibers and or fines are rather soft to the touch, making them an attractive choice as the stiffening agent. Other potential fibers can include carbon fiber, ultra high molecular weight (UHMW) polyethylene, cellulose, Nylon, polyester, fiberglass, polybenzoxazole (PBO), liquid crystal polymer fiber, polypropylene, polyamide, polybutyleneterepthalate, man made fiber, cotton, wood pulp and natural fiber. A buffer layer between the skin and the fiber base material can also be used to mitigate the rough feel of the fibers. Materials that can serve this function include wax coatings and thin thermoplastic laminate layers that are substantially fiber free.

### BRIEF DESCRIPTION OF THE DRAWINGS

FIG. 1 represents a radiation therapy patient immobilization mask made using fiber reinforced low temperature thermoplastic material.

FIG. 2 illustrates a hand splint made using fiber reinforced low temperature thermoplastic material.

FIG. 3 illustrates a nasal splint made using fiber reinforced low temperature thermoplastic material.

### DETAILED DESCRIPTION OF THE INVENTION

The material of the present invention can be a cross-linked thermoplastic polyester. Cross-linking can be employed to increase the body or viscosity of the polymer and can be achieved by subjecting the polymer to electron or gamma radiation in the range from 0.1 to 15.0 megarads. Alternatively, cross-linking can be achieved by subjecting the polymer to ultraviolet energy. An alternative method of cross-linking can be achieved chemically by the addition of an organic peroxide. The organic peroxide comprises between 0.1% and 10% by weight of the material.

Use of the discontinuous fiber and or fines can be adjusted depending on the desired use and characteristics. The length of the fibers can be up to 9 mm. In addition, several additives can be added depending upon the desired performance characteristics. For example, at least one additive can be added selected from the group consisting of silica, calcium silicate, cis-1,4 polydiolefin, ionomer, synthetic rubber, natural rubber, C. styrene-butadiene-styrene, glass spheres, glass micro balloons, phenolic spheres, phenolic micro balloons and styrene-isoprene-styrene triblock copolymers.

The present invention is particularly useful when used in the radiotherapy environment. State of the art cancer radiation therapy is increasingly based on the pinpoint application of high-energy radiation, which is highly tailored to the shape and position of the cancerous tumor. Modern techniques such as Intensity Modulated Radiation Therapy (IMRT) use a pencil sized beam whose cross-section is shaped to match the tumor. This allows the physician to spare the surrounding healthy tissue while increasing the treatment dose to the cancerous target. As the size of the treatment beam decreases, the accurate location of the beam becomes much more critical. If a highly tailored beam is off target by a few millimeters, it may miss the tumor entirely, destroying healthy tissue. Because of these new techniques, it is becoming increasingly important to ensure that the patient is positioned accurately and does not move during treatment. As these new treatments can take up to an hour, it is also imperative that the patient is relatively comfortable.

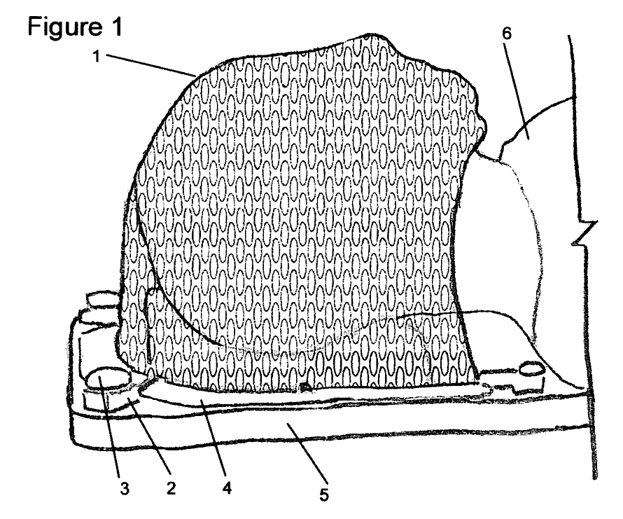

When used in radiotherapy patient immobilization, the fiber reinforced low temperature thermoplastic material would be molded over the patient's affected body part, such as the patient's head, to create a rigid shell. As shown in FIG. 1, the patient 6 is laying on a head immobilization board 5. The fiber reinforced low temperature thermoplastic has been molded over the patient's head, creating a rigid mask. The high temperature frame 4, which is bonded to the mask 1 is then secured to the immobilization board 5 with a turn screw 3 and a swivel lock 2.

In one embodiment the thermoplastic material would be bonded to a rigid frame. This thermoplastic and frame combination would then be heated in a 160 degree Fahrenheit water bath. At this temperature the thermoplastic becomes soft and very pliable. It is then removed from the water bath, towel dried, and then molded over that patient's head. This stretches the thermoplastic to approximately 250% of its original length. The rigid frame that is bonded to the thermoplastic would then be attached to the treatment table or an accessory to the treatment table (FIG. 1). In another embodiment, the softened thermoplastic would be attached directly to the treatment table or an accessory to the treatment table. In either of these embodiments it is very desirable for the finished thermoplastic mask to be as rigid and stiff as possible, as this helps prevent a twisting motion of the patient's head.

When used in applications where x-rays must pass through the material, it is desirable to minimize the attenuation of the material. This is particularly true in Radiation Therapy since high-energy x-ray beams can generate electrons as they pass through matter. This effect is known as Compton Scattering. Since the skin absorbs the electrons, severe skin damage can result. In order to minimize attenuation and Compton Scattering, it is important to reduce both the thickness and attenuation of the thermoplastic. By making the material stiffer, a lower thickness material can accomplish the same immobilization. By selecting fibers and fillers that are composed primarily of carbon, hydrogen and oxygen, absorption and Compton Scattering can also be reduced.

One of the limitations to radiotherapy patient immobilization is that it is desirable to have only low Z materials between the radiation therapy beam and the patient's skin in order to minimize Compton Scattering. As the photons from the radiotherapy beam pass through matter, electrons are given off which then impact the patient. Higher Z value materials cause more electron generation. Electrons do not penetrate the human body but are rather absorbed by the skin, causing skin damage. For this reason great care is taken in the design of thermoplastics used in radiotherapy to ensure that lower Z materials are used. Aquaplast is composed of Carbon, Oxygen and Hydrogen. Aramid fiber is made up from Carbon, Oxygen, Hydrogen, and Nitrogen. These elements are considered to be low Z materials. Thermoplastics frequently used in orthopedic applications, such as Polyform® and Orthoplast®, are not suitable for radiotherapy because they contain fillers such as talc (composed of Hydrogen, Magnesium, Oxygen and Silicon) and silica (composed of Silicon and Oxygen). Magnesium and Silicon are higher Z value materials and thus have a higher propensity of producing Compton Scatter and thus radiation skin damage if used within the treatment field.

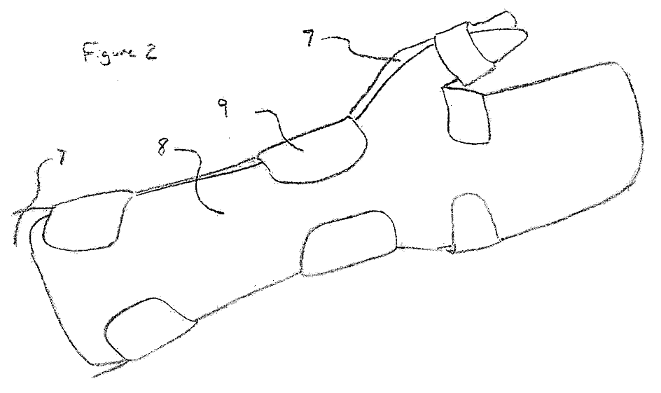

The present invention is also particularly useful in the area of orthopedic splinting or casting. Low temperature thermoplastics have been used for many years in the manufacture of custom splints, braces, and orthoses. Physical therapists, occupational therapists, hand therapists and orthotists soften the thermoplastic material in hot water and then mold it directly to the patients affected body part, creating a form that closely matches the anatomical contours of the patient's affected body part. The splint or cast is used to either immobilize the body part to allow for proper healing, to prevent a certain undesirable motion, or to promote a certain desirable motion. FIG. 2 shows the present invention with a patient 7 wearing a simple splint 8, known as a resting hand splint. Hook and loop strapping material 9 in also show in this figure. The purpose of this splint is to support the hand and wrist joint so that they heal without contracting and so that a deformity does not develop.

In any of these situations it is generally desirable for the custom splint to be as stiff as possible, as light as possible, and as thin as possible as the patient may have to wear the splint for several weeks. By increasing the stiffness of our current product via fiber reinforcement without materially increasing weight, a superior product is produced. It is also be possible to produce a thinner and lighter splint with the same stiffness as presently available materials.

This material can be used in the production of custom prosthetic socket cone production and/or reproduction. Increasing the stiffness of the material is a positive attribute when used in socket cone production and/or reproduction.

Custom heal cups can be made from cross-linked polycaprolactone as discussed in U.S. Pat. No. 5,415,623 to Cherubini. Short fiber reinforcement, as taught by the present invention, improves this product as it produces a stiffer product without an increase in weight. A thinner and lighter product can also be produced that has the same stiffness as the current un-reinforced product.

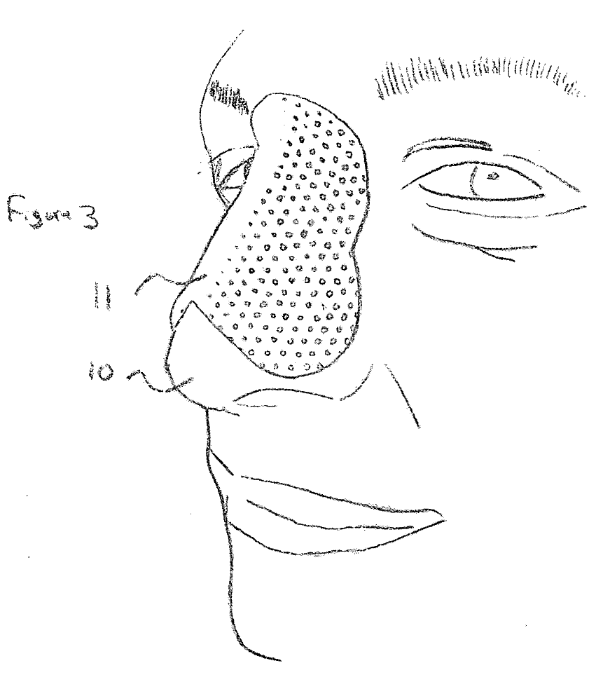

The present invention is particularly well suited for use in plastic and reconstructive surgery splinting. Low temperature thermoplastic nasal splints offer external stabilization and protection after a rhinoplasty or nasal fracture. They are softened in hot water and then molded over the reconstructed nose. As shown in FIG. 3, the patient 10 is wearing a nasal splint 11 die-cut into a shape developed by Dr. William Silver of Atlanta Ga. This splint is traditionally used after a rhinoplasty. The purpose of this splint is to displace the force of any accidental trauma to the nose that could potentially re-break the nose. They can then be secured in place with paper tape (or will bond to paper tape already applied to the nose). An adhesive film can also be applied to the material to aid in the bonding process. The primary purpose of this splint is to displace the force of accidental trauma to the nose that could potentially re-break the nose. It is important that the splint be as unobtrusive as possible (both physically and visually) as the patient must wear this splint for approximately one week. By increasing the stiffness of presently available unreinforced products via fiber reinforcement without increasing weight, a superior product is produced. It is also possible to produce a thinner and lighter nasal splint with the same stiffness as our current material.

We claim:

1. A cross-linked thermoplastic polyester having a melting point between 50 degrees Celsius and 85 degrees Celsius that is reinforced with a discontinuous short length fiber and or fines.

2. The material of claim 1 where the thermoplastic polyester is poly (epsilon-caprolactone) having a weight average molecular weight of greater than 5,000.

3. The material of claim 1 where the cross-linking is achieved by subjecting the polymer to electron or gamma radiation in the range from 0.1 to 15.0 megarads.

4. The material of claim 1 where the cross-linking is achieved by subjecting the polymer to ultraviolet energy.

5. The material of claim 1 where the cross-linking is achieved chemically by the addition of an organic peroxide and wherein the organic peroxide is benzoyl peroxide and the peroxide comprises between 0.1% and 10% by weight.

6. The material of claim 1 where the discontinuous fibers and or fines have a length of up to 9 mm.

7. The material of claim 6 wherein the fibers and or fines are at least one selected from the group consisting of aramid, carbon fiber, ultra high molecular weight (UHMW) polyethylene, cellulose, Nylon, polyester, fiberglass, polybenzoxazole (PBO), liquid crystal polymer fiber, polypropylene, polyamide, polybutyleneterepthalate, man made fiber, cotton, wood pulp and natural fiber and wherein the fiber comprises from 2% to 60% of the total material by weight.

8. The material of claim 1 further comprising at least one additive selected from the group consisting of silica, calcium silicate, cis-1,4 polydiolefin, ionomer, synthetic rubber, natural rubber, C. styrene-butadiene-styrene, glass spheres, glass micro balloons, phenolic spheres, phenolic micro balloons and styrene-isoprene-styrene triblock copolymers.

9. An orthopedic cast or splint made from the material of claim 1.

10. A radiotherapy patient immobilization device made from the material of claim 1.

11. A rigid dressing or nasal splint made from the material of claim 1.

12. A prosthetic socket cone production or reproduction made from the material of claim 1.

13. A custom heal cup made from the material of claim 1.

14. A thermoplastic polyester radiation therapy patient immobilization material having a melting point between 50 degrees Celsius and 85 degrees Celsius that is reinforced with discontinuous short length fibers and or fines.

15. The material of claim 14 where the thermoplastic polyester is poly (epsilon-caprolactone) having a weight average molecular weight of over 5,000.

16. The material of claim 14 where the discontinuous fibers and or fines have a length of up to 9 mm.

17. The material of claim 14 wherein the fiber and or fines are at least one selected from the group consisting of aramid, carbon fiber, ultra high molecular weight (UHMW) polyethylene, cellulose, Nylon, polyester, fiberglass, polybenzoxazole (PBO), liquid crystal polymer fiber, polypropylene, polyamide, polybutyleneterepthalate, man made fiber, cotton, wood pulp and natural fiber and wherein the fiber comprises from 2% to 60% of the total product by weight.

18. The material of claim 14 that is cross-linked in order to increase the body or viscosity of the polymer.

19. The material of claim 18 where the cross-linking is achieved by subjecting the material to electron or gamma radiation in the range from 0.1 to 15.0 megarads in order to cross-link the polymer.

20. The material of claim 18 where the material is cross-linked chemically by the addition of an organic peroxide.

21. The material of claim 20 where the organic peroxide is benzoyl peroxide.

22. The material of claim 21 wherein the benzoyl peroxide comprises between 0.1% and 10% by weight.

23. The material of claim 1 that has a wax coating.

24. The material of claim 14 that has a wax coating.

25. The material of claim 1 having one or more surfaces and a layer of thermoplastic material on at least one surface and wherein the thermoplastic material is substantially free of fibers.

26. The material of claim 14 having one or more surfaces and a layer of thermoplastic material on at least one surface and wherein the thermoplastic material is substantially free of fibers.
